(19) United States
(12) Patent Application Publication
(10) Pub. No.: US 2020/0147140 A1
(43) Pub. Date: May 14, 2020

(54) INTRAVENOUS ADMINISTRATION OF SUPRAPHYSIOLOGIC PLATELET RICH PLASMA FOR NEUROLOGICAL DISORDERS

(71) Applicant: Bridging Biosciences, LLC, Frisco, TX (US)

(72) Inventor: Tapley Holland, Frisco, TX (US)

(21) Appl. No.: 16/679,107

(22) Filed: Nov. 8, 2019

Related U.S. Application Data
(60) Provisional application No. 62/757,702, filed on Nov. 8, 2018.

(51) Int. Cl. A61K 35/19; A61K 35/16; A61M 1/02

(52) U.S. Cl. CPC A61K 35/19; A61K 35/16; A61M 2205/70; A61M 2202/0415; A61M 2202/0427; A61M 1/0281

(57) ABSTRACT

This invention relates in general to the field of cell-therapy treatments and more particularly, but not by way of limitation, to systems and methods for administering personalized cell-therapy treatments intravenously. In various embodiments, the system may calculate an aspiration volume needed for centrifugation to achieve a concentrated target threshold dose of 2×10⁶ platelets/μL for a particular cell therapy using various factors such as, for example, information about a patient and the efficiency of the concentration process.

### CROSS-REFERENCE TO RELATED APPLICATIONS

This patent application claims priority to U.S. Prov. Pat. App. Ser. No. 62/757,702, filed Nov. 8, 2018, which is hereby incorporated by reference for all purposes.

### BACKGROUND

### Technical Field

The present invention relates to treatment methods and protocols for the administration of supra-physiologic Platelet Rich Plasma (PRP).

### Background

The medical community has defined a normal circulating platelet count between the range of 150,000 platelets/μL, to 400,000 platelets/μL. Once circulating platelet counts go above 400,000 platelets/μL someone is considered to have thrombocytosis. Excessively high platelet counts are known to be a medical concern because it could lead to clotting which leads to stroke, particularly if it is an adult or if there is known arterial disease or prolonged immobility. Platelet counts that exceed greater than 1.0×10⁶ platelets/μL, are known as having secondary thrombocytosis and would be considered life threatening. Hence, the medical community has long considered excessive platelets above baseline to be a serious risk.

The use of concentrated platelet rich plasma (PRP) or concentrated bone marrow aspirate (cBMA) is widely understood and its clinical benefits are well established for a variety of tissue applications. The definition of PRP is a volume of blood plasma that has been centrifuged in order to produce a finite volume of PRP concentrate (PRP concentrate contains enriched quantities of different growth factors, platelets or thrombocytes, cytokines, exosomes, white blood cells, etc.) in order to stimulate healing tissue and remodeling. The definition of cBMA is a volume of whole bone marrow that is centrifuged to produce a finite volume of cBMA.

Historically the field of regenerative medicine has focused on disease conditions necessitating a site specific injection (hair, musculoskeletal, diabetic limb, etc.). In other words, injection of the therapy is typically done sub-dermally at the site of the injury or location of the pain. In recent years, there has been some reporting of intravenous administration of cells as a way to treat systemic diseases. These studies primarily report the use of stem cell infusion via a peripheral IV over an extended period time (5-30 minutes). Cell infusions typically include the mixture of saline, anticoagulants, or other cocktails to minimize the risk of clotting. While cells have no risk of clotting, platelets do, and it is difficult to void autologous cell therapies (like adipose or bone marrow) completely free of platelets during processing. Hence the reason why PRP, a high concentrations of platelets, has not been considered as an intravenous therapeutic strategy. The only known publication to attempt intravenous administration of PRP did not exceed 604,000 platelets/μL and only attempted it on pediatric patients. These platelet ranges were only slightly above normal and pediatric patients do not carry the risk of arterial disease. Hence, any cell therapy containing platelets, especially high platelet counts, would be considered against conventional wisdom.

For some therapeutic applications of PRP and cBMA, proven thresholds or doses have been established and verified. For example, it is generally agreed that an effective PRP concentration to stimulate angiogenesis is about 1.5×10⁶ platelets/μL administered directly to a location of an injury. Angiogenesis (new blood vessel formation) is often necessary for tissue genesis. Various scientific and research settings have established recommended doses for antimicrobial capabilities, pain relief, and other treatment applications. In the last several years, dosing for many autologous therapeutic treatments and tissue regeneration applications have been established. Furthermore, it has been observed that use of lower or higher concentrations of PRP is often less effective and commonly believed that use of excessively high concentrations has inhibitory effects.

Regenerative medicine relies on the power of the cells to perform activation, differentiation, and paracrine interaction to locally affect a diseased and damaged area (i.e., cell therapy knee injection for knee pain). PRP has an excellent safety record and can be obtained relatively noninvasively through a venous blood draw where the blood is then mechanically centrifuged to extract a concentrate of PRP. PRP has a long clinical use for tissue regeneration, reconstructive and plastic procedures, with perioperative administration facilitating wound hemostasis, sealing of wounds, augmentation of bone healing, and control of infection. The clinical risks of PRP concentrate injections at site locations are widely accepted as minimal for many applications.

In the field of regenerative medicine, dosing inaccuracy has stalled the progress of treatment discovery. In other words, without knowing the dose-to-outcome response, there is no way of knowing the treatment effects and benefits. While it has been shown that a minimum of 1.5×10⁶ platelets/μL stimulates new blood vessel formation and inhibition was shown at 3.0×10⁶ platelets/μL, this has only been demonstrated one time in the clinical literature for hair restoration. Hair injections is a site specific injection. When administering a site specific injection, conventional wisdom say to mix the platelets with an activating agent, like calcium chloride and/or thrombin, to lyse the growth factors and cytokines directly on the site injected. Furthermore, because site specific is not intravenous, it does not carry the risk of entering the blood stream and spontaneously clotting.

Many neurodegenerative diseases are characterized by loss of cerebral vascularization; endothelial dysfunction; epithelial tight junction disassembly; permeable or permissive intestinal, vascular and neuronal walls; gut dysbiosis, epigenetic modifications; cellular dysfunction or confusion; mitochondrial dysfunction; and exposure of neuronal axons resulting from demyelination. Clinical and experimental evidence has demonstrated the brain continually wants to repair; however, innate biologic repair is unachievable while under autoimmune and pathogen attack. While drug targeted treatments provide some level of symptomatic relief, they fail to address the underlying biology to promote remyelination and cellular repair.

Administration of PRP to transected sciatic nerve in animals has been shown to promote remyelination, suggesting that thrombocytes may offer a therapeutic solution for treatment of central nervous system disorders. Recent evidence in pediatric cerebral palsy patients demonstrated significant cognitive and language improvement compared to control patients. Additionally, evidence has demonstrated and established both angiogenic and bacteriocidal doses of PRP. So, not only do these cells provide a rich source of neurotrophic factors, they additionally play an active role in regulating innate immunity, angiogenesis, and provide a level of antimicrobial host defense. Derived from megakaryocytes, platelets are recognized to deliver exosomal payloads of anti-inflammatory mRNA and protein hyaluronidase activity capable of reversing chronic inflammation. Despite these patient benefits, PRP has failed to gain widespread clinical adoption due to the lack of quality control, standardization, and translation from the scientific to clinical setting.

To backfill this gap in understanding, Applicant has developed a method of quality control and cell software capable of calculating exact aspiration volumes to deliver consistent platelet numbers (dose) to patients in need of treatment. Applicant's Harbour Cell Software™ follows the following exemplary steps:
a. Test baseline platelet count via finger stick (capillary sampling);
b. Calculate exact aspiration volume needed for centrifugation to achieve target dose;
c. Test PRP post-centrifugation to confirm requested target dose;
d. Calculate re-aspiration or dilution volumes to adjust the target dose; and
e. Verify the target dose/mL has been produced prior to treatment.

Several inherent variables influencing cell quantities throughout the treatment process and protocol have been identified. For example, the clinical preparation of PRP is largely pre-defined by the commercial centrifuge kit being used. However, the respective cell recovery performance efficiency of these machines varies greatly. Known aspiration techniques are often labor intensive and time-consuming to execute. In addition, varying degrees of cell yields per μL may be achieved when using different syringe sizes, styles of aspiration needles, and/or duration of the aspiration. Utilizing Applicant's Harbour Cell Software™ may help reduce some of these variabilities.

Currently, the FDA has outlined 24 diseases that would be considered of unmet need with greater that 71% falling into the category of autoimmune, auto-inflammatory and chronic diseases (“AAC”). Fibromyalgia, chronic fatigue syndrome, alopecia areata, autism, gastrointestinal disorders, etc. are a few examples of these diseases. Not only are there usually no known cures, but these diseases have crippling effects on quality of life and are an annual economic healthcare burden in the trillions of dollars. These conditions fall into the category of having multifactorial underlying causations collectively marked by, for example: endothelial dysfunction; epithelial tight junction disassembly; permeable or permissive intestinal/vascular and neuronal walls; gut dysbiosis, epigenetic modifications; cellular dysfunction or confusion; mitochondrial dysfunction; and exposure of neuronal axons resulting from demyelination.

These causations are often the result of evolved harmful invaders (pathogens, viruses, and harmful bacteria) breaking down the body's immune defense system and manipulating it to their advantage for survival. The understanding of invaders to manipulate bodies to their advantage has been studied extensively—particularly breaking down or finding weak spots in protective walls, commonly referred as the blood-brain barrier and the gut-brain barrier. Once these walls are breached, invaders enjoy the battle advantage and engage the immune system with an array evolved measure/countermeasure tactics for system manipulation. These invaders are known to recruit cells, lodging inside a cell or organ to avoid removal, manipulate a cell or organ from the inside out, and hijack neurotransmissions and cell communications to gain precious information about response and adapting. When invaders outnumber the immune defense, a tipping point occurs leading to the multifactorial causations of an AAC disease.

The body's immune system and immune defense against these invaders is heavily dependent on the gut microbiome. The human microbiome outnumbers the body's cells in our body by 10:1 and consists of genes 150 times larger than the human genome. From birth and beyond, gut microbes are constantly communicating directly with gut epithelial and immune cells in an orchestrated process of protection, communication, and learning—including how to distinguish host from invader. The goal of invaders is to escape the enclosed walls of the intestinal gut and the vasculature to roam freely in the body, whereby seeking out the various organs of the endocrine and central nervous system. To maintain the battle advantage, the body must ensure walls are tightly enclosed to help corral invaders for the body to target and remove. The microbiome is a critical part of controlling access through these protective walls and thus is constantly under attack and manipulation from invaders.

Fibromyalgia is a classic, chronic, incurable AAC disease example that demonstrates all of these things mentioned. Patients present with severe musculoskeletal pain, general fatigue, loss of sleep, brain fog, loss of cognition, as well as, mental health decline. In the U.S., 20 million people are diagnosed each year costing the healthcare system an average of 280 billion dollars annually. It is now known that the underlying pathophysiology of fibromyalgia is directly linked to altered gut bacteria. In this case, altered bacteria have learned to survive the harsh environment of the gut bile, while simultaneously secreting neurotoxins or substances to permeate the gut. At this point, every individual is at the mercy of their immune system engaging in a battle of measure and countermeasures. For some unfortunate humans, a tipping point is reached whereby the invaders outnumber and overtake the body's immune system to begin system manipulation and rewiring.

Platelets represent a medicinal warehouse of growth factors, peptides, neurotrophic factors, and cytokines to address the underlying causations of AAC diseases as well as stimulate the appropriate repairs. Traditionally, the primary platelet function is hemostasis, however, it is now understood that platelets function more like autonomous drones circulating the body executing immune surveillance, invader detection, coordinating attacks, stimulating tissue repair, and searching for breached wall integrity. To execute these functions, platelets rely on growth factors contained within them that are specifically responsible for: promoting new blood vessel formation; remyelination of nerves; immunomodulation; re-epithelialization; reendothelialization; and microbicidal properties—making the platelet the ideal regenerative specialist that is capable of restoring function (both communication and operational) to dysfunctional cell, as well as initiate the specific repairs associated with AAC diseases that are needed for healing.

PRP is an autologous concentrate of platelets and growth factors within a relatively small volume of blood plasma intended to promote healing through the restoration of tissue function. PRP is prepared through a process known as differential centrifugation, in which the acceleration force applied to the blood sample during centrifugation fractionates the cellular constituents based upon differences in specific gravity, whereby platelets are often the lightest cellular fraction. PRP is an autologous cell therapy that has been popularized over the last three decades through thousands of published clinical studies and case reports describing its remarkable regenerative potential. Despite its potential, past studies are limited by the absence of tools enabling the point-of-care standardization of the delivery of pre-determined platelet densities to patients in need of treatment. Thus, scientific and clinical discovery has stalled in the field of regenerative medicine due to dosing inaccuracy (overdosing or underdosing) and the inability to understand the dose-to-outcome response for various treatments.

### SUMMARY OF THE INVENTION

This invention relates in general to the field of medical treatments. The present invention relates to treatment methods and protocols for the intravenous administration of a supra-physiologic Platelet Rich Plasma (PRP) therapy dose for neuroendocrineology, neurorehabilitation, neuronal degenerative, neuronal sensory, and neurocognitive diseases. More specifically, various embodiments of the described treatment methods may utilize Applicant's Harbour Cell Software™ to enhance treatment results by determining a pre-centrifugation aspiration calculation. The current treatment method describes implementing the aforementioned cell therapy device to calculate aspiration volumes in order to achieve a minimum of 2.0×10⁶ platelets/μL, of injection. The purpose of the cell therapy device is to help clinicians deliver consistent cell therapy doses to patients, whereby a patient specific aspiration calculation is determined by a pre-determined concentration target, thus ensuring an element of quality control and standardization that currently does not exist in regenerative medicine. Standardization of regenerative medicine leads to knowledge advancement and the discovery of new treatments and protocols.

One such discovery is the use of PRP to treat AAC diseases. Because invaders outnumber defenses in AAC diseases, it is therefore necessary to introduce platelets in high concentrations—i.e., PRP. Furthermore, because invaders enjoy the freedoms of navigating throughout the body, intravenous administration of PRP would conceivably be the ideal therapeutic strategy instead of local site injection. To successfully treat an AAC disease with intravenous PRP, it is necessary to determine the dose to outcome and the method of PRP preparation. Applicant has developed several testing tools, processing tools, and artificial intelligence (AI) learning software that collectively personalize cell therapies to individual patients using Applicant's software described U.S. Pub. Pat. App. No. 2019/0088372, which is hereby incorporated by reference. Applicant's artificial intelligence software uses various proprietary learning metrics and algorithms to learn a dose to outcome relationship of a respective cell therapy. The spirit of the collective patents is aimed to provide physicians and scientists a standardized methodology for finding regenerative medicine treatment protocols. In the context of this patent, these tools and software were used within an IRB study to treat over 100 patients suffering from AAC diseases. Shortly into the study, a minimum therapeutic dose to outcome relationship was learned and a treatment protocol for the remaining patients suffering an AAC disease was born.

In various embodiments, a method and treatment protocol is provided for obtaining and administering a dose specific therapy of PRP, intravenously, for neurological disorders. More specifically, software may be used to calculate a patient-specific blood-aspiration volume to produce the minimum treatment dose for neurological improvements at 2.0×10⁶ platelets/μL. In some embodiments, artificial intelligence may be used to improve accuracy. The method describes intelligence software used in conjunction with testing equipment to determine the patient-specific blood calculation, as well as the treatment protocol of intravenous administration. The artificial intelligence software is intended to learn the intended dose for specific disease conditions. Various embodiments have resulted in unexpected findings and discoveries resulting from using the system and method described herein and in testing various components thereof

In some embodiments, an intravenous administration of a minimum of 2.0×10⁶ platelets/μL, is administered without an activating agent or dilutant. Following centrifugation and confirmation of target dose, the treatment syringe needle may be inserted directly into a patient's vein, such as an arm vein, and administered at a rate of approximately 1 mL/second. The dosage in the syringe was not diluted with saline or any anticoagulants to prevent clotting, nor were any activating agents added. In test cases, the dose was administered to patients that are not considered pediatric, 84% of the patients being over 40 years of age. In 50% of the patients, the dose exceeded 3.0×10⁶ platelets/μL. Surprisingly, against conventional wisdom and thought, the treatment protocol described herein achieved therapeutic benefits via direct administration of supraphysiological concentration of PRP.

The results Applicant has achieved prove the novelty of the intelligence software and ultimately the novelty of the treatment methods described herein. Dosing inaccuracy and lack of standardization is a widely known problem within the scientific literature, especially PRP. Because there has been a lack of tools to help prepare consistent dosing, treatment protocol discovery has stalled in the regenerative medicine field. Reducing Applicant's Harbour Cell Software to practice led to unexpected clinical findings and the discovery of novel neurological cell therapy treatment protocols, including supraphysiologic dose administration intravenously of PRP. In this 100-patient study, 100 patients presented with an AAC disease and, after receiving the novel treatment protocols described herein, of the 100 patients, 92% reported an improvement in quality of life surveys. In 49% of them, this item was evident with statistical significance (p<0.005). In those patients with neurological tare an improvement in the cognitive sphere was observed in 70% of them (greater capacity for concentration, language or ability to perform more complete tasks) with statistical significance (p<0.002).

To date, no reports within the clinical literature can be found investigating, demonstrating, or reporting these similar clinical findings. Ultimately, these findings were initially unexpected and resulted in the discovery of a consistent therapeutic dose for AAC disease patients. There are no reports characterizing platelet dosing of PRP-IV treatments for neurological disorders.

The above summary of the invention is not intended to represent each embodiment or every aspect of the present invention. Particular embodiments may include one, some, or none of the listed advantages.

### BRIEF DESCRIPTION OF THE DRAWINGS

A more complete understanding of the method and apparatus of the present invention may be obtained by reference to the following Detailed Description when taken in conjunction with the accompanying Drawings wherein:

FIG. 1 is a flowchart of a method according to an embodiment; and

FIG. 2 is a flowchart of a method according to an embodiment.

### DETAILED DESCRIPTION

The present invention is directed towards systems and methods for increasing successful outcomes in cell therapy treatments. This treatment protocol may include the use of Applicant's Harbour Cell Software™ or other device to calculate exact aspiration blood volumes in order to concentrate a consistent PRP minimum dose on the order of 2.0×10⁶ platelets/μL, for neurological disorders. Furthermore, the PRP is administered intravenously to promote circulatory repair.

One novelty of this treatment protocol includes delivering a consistent PRP dosage to all patients. The Harbour Cell Software™ may be used to determine the exact blood aspiration volume in order to achieve consistency of PRP dosing. First, a baseline platelet count was taken from each respective patient. Next, the baseline platelet count, the centrifuge recapture efficiency, injection volume, and target dose of on the order of 2.0×10⁶ platelets/μL, were all used to calculate and display exact aspiration volumes for each patient. The aspiration volumes were highly variable for each patient, but a minimum of 2.0×10⁶ platelets/μL, was achieved for this embodiment. In other embodiments, the concentration may be higher or lower than 2.0×10⁶ platelets/μL, may be utilized depending on the treatment parameters. The 2.0×10⁶ platelets/μL, dose was found to be sufficient to accomplish both bacteriocidal and blood vessel formation, which are needed for autoimmune/inflammatory neuro-related diseases.

Another novelty of this treatment protocol was the intravenous administration of the PRP. PRP is widely known and administered via a local injection to an injured site. Local administration has the drawbacks of prematurely releasing platelet growth factors and peptides, whereas the benefit of the IV administration is the systemic release during circulatory repair. During one study performed by Applicant, nine of the eleven patients received IV administration of surpraphysiologic PRP. All nine of these patients reported improvements to neurocognition, language, memory, eyesight, handwriting, focus, systemic pain relief, inflammation reduction, detoxification, and academic grade improvements. The two patients who received localized injections only reported pain relief at their injured sites. These two patients did not report any other improvements like those reported from the patients receiving the IV treatment.

Various embodiments are directed towards a cell therapy treatment protocol for neurological disorders. More specifically, a treatment protocol administering, on the order of, at least 2.0×10⁶ platelets/μL, intravenously. In various embodiments, the treatment protocol calls for consistency of platelet dose administrations, which may be achieved by following the systems and methods of, for example, applicant's Harbour Cell Software™ to calculate exact blood aspiration volumes to achieve consistent doses.

Currently, point-of-care cell therapy lacks sufficient standardization. In various embodiments, the systems and methods may utilize the Harbour Cell Software™ for making the latest treatment protocols available to doctors, nurses, and other technicians at the point-of-care. When conducting a cell therapy treatment study in a controlled setting, several safety measures may be in place to ensure accuracy that may not be in place in a real-world setting. In both the research and real-world settings, cell therapy treatments generally include a physician aspirating a determined large volume of autologous fluid from a subject, concentrating this fluid via centrifugation to obtain a final small volume of concentrate, and then injecting this small volume concentrate to a target site. In this treatment protocol embodiment, the final small volume of concentrate is delivered intravenously for circulatory repair.

Clinical outcomes are more likely to succeed when a target concentration is achieved prior to centrifugation. Furthermore, the advancement of cell therapy regenerative medicine can occur when methods and devices insuring quality control and dose consistency are understood and administered. In order for one to be sure enough total cells are present to centrifuge, despite all of the other variables, one must aspirate the appropriate volume from the patient to reach a desired target platelets/μL, in the final volume of concentrate. Physicians often determine their aspirating volume by the centrifuge kit volume limitations, habitually aspirate the same volumes for each patient, or stop aspirating when they feel they have enough.

In various embodiments, systems and methods utilizing the Harbour Cell Software™ ensure the appropriate amount of target platelets/μL, has been achieved in the final small volume of concentrate. In the present embodiment, neurological improvements were achieved by delivering consistent target platelets/μL, intravenously.

The underlying manifestations that affect neurodegenerative, sensory, cognitive, rehabilitation, and endocrine disorders are multifactorial and complex. However, an appropriately dosed PRP therapy may offer new hope in addressing its causation. Epitheilial, endotheilial, and PRP-derived growth factors could address intestinal and vascular permeability causations, as well as, revascularization. Phagocytotic PRP peptides and macrophage reprogramming could not only restore gut dysbiosis, but remove free roaming neurotoxic bacteria, pathogens, and viruses. Furthermore, phagocytosis would address cell-invading small-colony variant bacteria capable of cell mimicry and disruption of cell mitochondria. Remyelination of neuronal axons, via brain derived neurotrophic factor, could restore connectivity and axonal protection from free roaming bacteria/pathogens and viruses. Lastly, PRP/macrophage induced inflammation would signal an orchestrated tissue repair remodeling cascade (tissue genesis) via paracrine signaling and cell proliferation/differentiation choreographed via platelets and growth factors. Various embodiments of treatment methods may include using Applicant's Harbour Cell Software™ to ensure consistent and accurate results, discovery and refine new treatment methods, and/or supraphysiologic dose administration intravenously of PRP.

To date, nothing within the clinical literature can be found investigating, demonstrating, or reporting the clinical findings described herein and, ultimately, the discovery of a treatment method for intravenous administration of PRP for neurological disorders. Furthermore, there are no reports characterizing consistent platelet dosing of PRP-IV treatments for neurological disorders.

In various embodiments, the treatment method may include a testing protocol, AI dose learning, and/or an intravenous administration protocol of the PRP. In a first step, various patient demographics may be inputted into the software for learning. Demographics can include, but not limited to, disease state information, health information, objective patient characteristics and lifestyle information. For example, the medications a patient is taken, the patient's history of viral infections, and/or consumption of alcohol, all of which may affect platelet counts, may be inputted. Demographic information may also include genomic and genetic specifics of individual patients. This information is compared against the learning database to determine the degree of impact each demographic variable potentially has on outcome success. Over time, the learning metrics understand the degree of each independent variable impacting outcome success which is then correlated back to dosage.

In a second step, a patients baseline platelet count is established, such as via finger stick (capillary sampling). Following the inputting of demographic information, a capillary sampling of blood may be executed. The blood sample could be sampled and counted different ways known to those skilled in the art. In some embodiments, blood sampling testing kits may be used that contain specific capillary tubes and testing slides to increase accuracy.

In a third step, the exact aspiration volume for centrifugation is calculated. Once the demographic information and baseline platelet count have been inputted into the software, a blood aspiration calculation may be determined and displayed. The learning software may accounts for demographic variability, baseline cell counts, and/or previous recorded outcomes to determine an overall dose-to-outcome relationship. For example, the software may have learned that a minimum dose/mL is needed for success of a treatment for a certain disease. For a patient presenting with that disease and having several demographic variables that impact baseline cell counts, the software may be able to adjust accordingly. Baseline cell count is one of several variables that impact dosing inaccuracy. Additionally, other variable can impact the calculations. For example, individual operators (human error) have different variabilities of performance that are often unavoidable. The artificial intelligence software may learn individual operator performance deviations and may include that in the various metrics factored into patient aspiration calculations. Based on these collective learning metrics and algorithms, the software may then calculate the patient-specific blood aspiration volume needed to produce the minimum consistent therapeutic dose/mL for the disease treatment.

At a fourth step, target dose is tested post-centrifugation for re-aspiration or dilution adjustments. Following centrifugation, the testing protocol may call for confirmation of the target dose. A sample of the final concentrate is collected and a measurement is initiated. Once the final measurement is inputted into the software, dilution calculations or re-aspiration calculations are performed. By way of example, in real-world patient encounters, Applicant's software has achieved better than 95% accuracy in calculating a blood aspiration that will produce a desired target dose.

At a fifth step, re-aspiration for centrifugation or dilution adjustments to reach desired target dose are performed. It is often easier to dilute than to re-aspirate, re-centrifuge, homogenize to final concentrates, re-test and verify. Therefore, the learning metrics can be defaulted to err on the side of over aspiration to avoid those time-consuming processes. At a sixth step, the treatment therapy is administered. At a seventh step, patient follow up and documentation of validated outcomes are measured. The determination of a dose-to-outcome response is dependent on the outcome success of the treatment. Outcome success is collected by the scientific and medical community via validated measurements scales depending on the disease and condition. For example, the pain VAS measurement scale is a unidimensional measure of pain intensity, which has been widely used in diverse adult populations for various treatments and outcome measurements. The change in a patient's pain measurement is an example of a successful treatment outcome metric that may be incorporated into the learning algorithms intended to learn a dose-to-outcome response.

Using the learning software, Applicant has discovered new dosages for treating AAC diseases. Applicant conducted a series of validation studies to satisfy FDA requirements. An Institutional Review Board (IRB)-approved validation study was executed to originally only include 10 patients. The first patient, and each subsequent patient, consented and elected to have the PRP intravenously administered due to various ailments all related to AAC diseases. After demographic data was collected, a licensed, board-certified physician auto-transfused the PRP using guidelines set forth by the American Society of Hematology (ASH). The first few patients reported remarkable and similar improvements related to neurocognition, sensory, communication and other metabolic changes. Unexpectedly, due to these initial results, patient word of mouth turned the small validation study into 100 patients being treated. All patients fell into the category of having an AAC disease. All patients had exhausted conservative treatments and, seeking an alternative treatment, consented to IV PRP administration. Thus, Applicant initiated an outcome survey questionnaire for the purposes of learning a successful dose-to-outcome response and neurological exams for learning the dose for individual improvements.

In one embodiment, a quality of life questionnaire was given and neurological assessment measurements were taken. These validated measurement scales were used to determine positive response and metrics from these responses were correlated with PRP dosage. In certain variations, several validated measurement scales could be used in a similar manner to correlate positive outcomes success to minimum dose. Other quality of life surveys or neurological measurement exams can be used and the software can accommodate the inputting of various scales for learning.

In another embodiment, the learning algorithm correlates the various positive metrics of the outcome scales to the various PRP doses delivered. The software may be designed to determine a dose-to-outcome response with greater than 90% confidence. From the 100 patients treated, the software determined 2.0×10⁶ platelets/μL, was the minimum therapeutic PRP dose needed for patient positive response. In certain variations, the % confidence of learning software may comprise of a range anywhere from 1-100%.

In another embodiment, the preparation of the PRP may involve a blood phlebotomy and centrifugation. The blood phlebotomy may be executed using World Health Organization guidelines via a butterfly needle, tourniquet, stopcocks, and multiple syringes. The centrifugation preparation process may involve a two-step centrifugation process for PRP sedimentation. In order to produce a supraphysiological concentration of platelets per μL may require a two-step process. Many clinicians only execute a one-step centrifugation protocol to produce PRP, which is often not capable of producing high concentrations of platelets. There are many two-step PRP processing kits commercially available. For example, the Pure PRP system from Emcyte Corporation is a commercially available PRP kit that involves a two-step preparation process. The Pure PRP system first uses a canister for RBC and platelet supernatant sedimentation. Following the first centrifugation spin, the operator withdraws the lighter supernatant and injects this into the second and final processing canister for separation. The second centrifugation spin separates the platelet supernatant into platelet poor plasma and platelet rich plasma. Once centrifugation is finished, the operator removes a volume of platelet poor plasma and continues by homogenizing the remaining volume. The volume of platelet poor plasma can be a various percentage depending on the final volume of PRP needed for injection. The percentage can be anywhere from 1-95% of the total volume being removed. Depending on the calculated blood amount, either one or two cylindrical canisters may need to be filled with an appropriate amount of blood. If only one canister was used, a counterweight may be used to balance the first canister. In either scenario, the first centrifugation spin may be executed for two minutes at 3800 rpm to isolate red blood cells and platelet supernatant. The platelet supernatant may then be removed and placed in a third canister for isolation of platelet poor plasma and platelet rich plasma. Whether a single or double canister, the second spin may be executed for seven minutes at 3800 rpm to produce a final PRP concentrate of a minimum 2.0×10⁶ platelets/μL. Following centrifugation, a small aliquot of PRP may be removed to confirm a 2.0×10⁶ platelets/μL, dose. In trial cases, the minimum 2.0×10⁶ platelets/μL, was achieved and an injection volume of 7 mL was infused to patients intravenously at 1 mL/second. In other cases, the concentration was increased to 2.5×10⁶ platelets/μL, and in other cases, to, on the order of, 3.0×10⁶ platelets/μL, or higher. In some embodiments, the dose was administered at 0.5 mL/second or lower, while in others it was administered at 1.5 mL/second, 2.0 mL/second or higher. In some embodiments, the injection volume was between about 6 mL to 8 mL, while in others it was reduced to below 6 mL, while in others it was increased to above 8 mL.

In some variations, the PRP preparation protocol could be a variation of centrifugation times and forces for both the RBC sedimentation and PRP sedimentation. Times can range anywhere from 1-20 minutes or more depending on the step of the process. Forces can range anywhere from using gravity to a centrifugation force up to 100,000 rpm. The time needed for sedimentation in the two steps would be proportional to the forces being used.

In some variations, the PRP processing components can be a variety of shapes and sizes. PRP processing components can also be a commercially available PRP kit or a combination of assembled components. For example, a minimum 2.0×10⁶ platelets/μL dose may be achieved with laboratory conical tubes. The conical tubes may require a larger calculated amount of blood draw, however, a two-step centrifugation process may still apply, as well as, determined time and centrifugation force. In either scenario, the performance of the commercial processing kit or manual processing method is one of the variables of blood aspiration calculations. The learning software algorithms learn the standard deviation of performance for the method being used to help calculate consistent PRP doses.

In some variations, the PRP preparation process involves a human operator whereby introducing human error. Any additional errors affect blood aspiration calculations which ultimately impact consistency of the dose. For example, a first operator could add an additional 10% of error compared to a second, more skilled, operator. The learning software metrics learn the proficiency of different operators as another variable influencing the consistency of dosing.

In another embodiment, a small aliquot sample is used for measurement to determine the minimum 2.0×10⁶ platelets/μL, has been achieved. Following centrifugation and homogenization of final concentrate sample, a transfer cup may be used to procure, for example, a 5 μL sample for analysis and measurement. The volume of measurement could be various volumes depending on the measurement instrument being used.

In another embodiment, a small aliquot sample is used for measurement to determine the patient's baseline platelet count. The baseline platelet count is another variable factored into the intelligent aspiration calculations. For example, a 20 μL capillary sample may be taken and used for measurement. The volume of measurement could be various volumes depending on the measurement instrument being used.

In some variations, the PRP compositions of each individual may comprise of varying concentrations of various types of white blood cells, lymphocytes, monocytes, eosinophils above their respective baseline counts. These concentrations over baseline are typically reported as “times baseline.” For example, the concentrations may vary between 1×-10× over their respective baseline. The concentrations of lymphocytes and monocytes may be between about 1.1 and about 2 times baseline, about 2 and about 4 times baseline, about 4 and about 6 times baseline, about 6 and about 8 times baseline, or higher. The concentrations of eosinophils in the PRP composition may be about 1.5 times baseline. In some variations, the lymphocyte concentration is between about 5,000 and about 20,000 per μL and the monocyte concentration is between about 1,000 and about 5,000 per μL. The eosinophil may be between about 200 and about 1,000 per μL.

In certain variations, the PRP composition may contain a specific concentration of neutrophils. The concentration may vary between less than the baseline concentration of neutrophils to eight times the baseline concentration of neutrophils. In some variations, the neutrophil concentration may be between 0 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration may additionally or alternatively be specified relative to the concentration of the lymphocytes and/or the monocytes. In preferred embodiments, the neutrophil concentration is less than the concentration in whole blood. In other embodiments, the neutrophil concentration is 0.1 to 0.9 the concentration found in whole blood, yet more preferably less than 0.1 the concentration found in whole blood. In other embodiments, the neutrophils are eliminated or non-detectable in the PRP composition.

The results from this study proves the novelty of the intelligence software and ultimately the novelty of the treatment methods described herein. Dosing inaccuracy and lack of standardization is a widely known problem within the scientific literature, especially PRP. Because there has been a lack of tools to help prepare consistent dosing, treatment protocol discovery has stalled in the regenerative medicine field. Reducing Applicant's Harbour Cell Software to practice led to unexpected clinical findings and the discovery of novel neurological cell therapy treatment protocols, including supraphysiologic dose administration intravenously of PRP. In this 100-patient study, 100 patients presented with an AAC disease and, after receiving the novel treatment protocols described herein, of the 100 patients, 92% reported an improvement in quality of life surveys. In 49% of them, this item was evident with statistical significance (p<0.005). In those patients with neurological tare an improvement in the cognitive sphere was observed in 70% of them (greater capacity for concentration, language or ability to perform more complete tasks) with statistical significance (p<0.002).

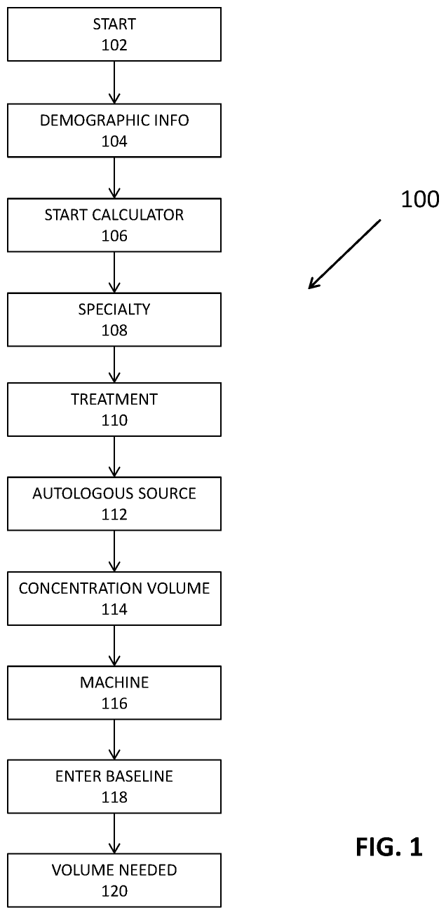

Referring now to FIG. 1, a flowchart is provided of an embodiment of a method 100 of providing a treatment protocol using a user device. At step 102, a user is prompted to select whether the use will be for research purposes or for non-research purposes. In various embodiments, at step 104, patient information and/or de-identified demographics of the intended subject could be inputted or selected from a drop down menu, such as, for example, whether the subject is human or animal, the sex of the subject, age, name, initials, or other indicia, treating physician, facility, location, and other relevant information. Such information may be useful for tracking, data and research collection purposes. These selections may be inputted and displayed via the user device.

At step 106, the user begins the calculation workflow. At step 108, the user device receives input from the physician regarding specialty. If being used for non-research purposes, then the user device may be programmed to prompt specialty selections that are within the published literature showing cell therapy human outcomes within the specified specialty. If the specialty is not reported in published literature or not published with human outcomes, then, at step 108, the physician may be prompted to add the specialty before proceeding through the workflow prompt. When a new specialty is added, the physician may be notified by the user device that it will no longer be accessing the cloud based and/or embedded published outcomes. The device may still proceed through the workflow prompts and calculate the needed autologous volume, however, the physician may be prompted that the volume calculated is intended for an experimental specialty use not reported in the published literature.

In the current treatment protocol embodiments, the physician specialty would fall under those pertaining or who treat neurological disorders (i.e., neurologist, neurosurgeon, rheumatologist, etc.). The Harbour Cell Software™ may be utilized for research purposes, and thus falling into the category of not reported in published literature.

At step 110, the device receives input from the physician regarding an intended treatment. If being used for non-research purposes, then the device may prompt treatment selections based at least in part on the specialty selection that are within published literature showing cell therapy human outcomes. If the intended treatment is not reported in published literature or not published with human outcomes, then the physician will be prompted to add the treatment before proceeding through the workflow prompt. If a treatment is added, then the physician may be notified by the machine that it will no longer be accessing the cloud based and/or embedded published outcomes. The device may still proceed through the workflow prompts and calculate the needed autologous volume, however, the physician may be prompted that the volume calculated is intended for an experimental treatment use not reported in the published literature. The physician may need to acknowledge this before proceeding to the next workflow prompt. If the device is being used for research purposes, then the physician may input the designated specialty.

In the current treatment protocol embodiment, IV dose administered PRP treatment is not reported in published literature and therefore must be inputted into the software before proceeding with the workflow prompts.

At step 112, the device receives input from the physician regarding an intended autologous source and strength number. The Harbour Cell Software™ incorporates these attributes as part of the workflow so physicians can be informed of the most current published methods during the course of treatment workflow. In the current embodiment, this treatment protocol discovery falls into the experimental treatment workflow prompts that are not reported in literature. The autologous source entered is autologous blood-PRP.

At step 114, the device receives input from the physician regarding concentration volume needed. If being used for non-research purposes, the device may only prompt a default numerical concentration milliliter volume, determined from the treatment selection. The defaulted volume may be based at least in part on the relevant published literature. The treatment targeted cell range per μL is displayed for the physician to view and confirm. In the current treatment protocol embodiment, a minimum of, on the order of, 2.0×10⁶ platelets/μL, was inputted as the target as there is no established target range of neurological disorders. Considering many neurological disorders require phagocytosis of various pathogens and bacteria, as well as, re-establishing blood vessel formation (neo-vascularization), the current target dose was chosen to encompass both established angiogenic and bacteriocidal in-vitro dose capabilities. The physician may have the option to increase or decrease the concentration volume by selecting plus (+) and minus (−) signs. The value for the starting volume needed to achieve the concentration volume needed for treatment is calculated based at least in part on the different inputs received during the workflow. Any number of calculations can be used to determine starting volume needed to maintain target platelets/μL, necessary for the intended treatment. Changing various inputted values will affect the resulting calculations.

At step 116, the device receives input from the physician regarding the concentration machine being used. The performance criteria is another example of a value that may influence the starting volume calculation. This is due to the known studied performance variabilities of commercial cell concentration devices. The physician may have options to add a machine. When adding a machine, a weighted performance average may be calculated to determine the starting volume calculation. In this scenario, the device may notify the physician that a weighted average is being used to determine the final calculations and not a known performance for the added machine. In the current treatment protocol embodiment, an established commercial centrifuge was utilized with similar performance characteristics and weighted average calculations to other known commercial centrifuges. These machine performance criteria were utilized to determine the final aspiration calculations to achieve consistent PRP concentrate yields of 2.0×10⁶ platelets/μL, for this particular concentration device.

At step 118, the user device receives baseline cell numbers that will be used for calculations. The baseline cell number can be taken by any commercially available cell counter, platelet counter, hemocytometer, or like device. The user device may receive the baseline numbers via manual input or via wired or wireless connection to the counter and/or other backend system to determine which calculation should be performed: for platelets, RBCs, HSCs, WBCs, exosomes, adipose pre-cursor cells, or MSCs. The user device can also be connected, either wired or wirelessly, to capable cell counting devices in order to transfer baseline data instead of manual input. These selections are displayed and received via the user device. Information provided by the system based at least in part on user inputs may assist the user in determining the source material to be used in the treatment. Because the baseline may vary depending on the source material, in various embodiments, although not required, it may be preferable for a user to input other information (e.g., specialty, treatment, and/or autologous source) before determining and/or inputting the baseline cell number. In the current treatment embodiment shown, the baseline platelet number is inputted after various other information has been entered.

At step 120, the device displays the starting volume amount of autologous source volume needed to be aspirated from the subject. This final calculation is determined based at least in part on the previous inputs from the clinician. This starting volume is the final calculated volume needed from the individual subject, to be concentrated, in order to concentrate a final treatment volume containing a minimum of 2.0×10⁶ platelets/μL, range.

In some embodiments, the device may also determine dilution and/or hyperconcentration calculations. Dilution and/or hyperconcentraton calculations may be an important value to know following machine concentration. By way of example, in various embodiments of the system, the starting volume was calculated to ensure an appropriate target platelets/μL, treatment yield is achieved. Following machine concentration, a physician can test a small aliquot sample of the concentrate. If the platelets/μL, volume exceeds the intended target, the excess plasma or other fraction of the separation can be added to dilute the concentrate in order to achieve the intended target platelets/μL, The treatment system may calculate exactly how much dilution should be added. The opposite would occur if too little target platelets/μL, were achieved. If the target platelets/μL, is less than the intended target treatment need, then hyperconcentration would need to occur. In this case, a cell analysis of the concentrate would be used by the system to make the hyperconcentration calculations. The system would calculate the amount of excess plasma or separated fraction to be removed from the concentrate. This would yield a total volume less than the desired treatment volume, but would be at the desired treatment target platelets/μL. Any number of weighted algorithms and calculations could be used to determine the final volume needed or other values within the equation. In the current treatment protocol embodiment, a minimum of 2.0×10⁶ platelets/μL was achieved before administering.

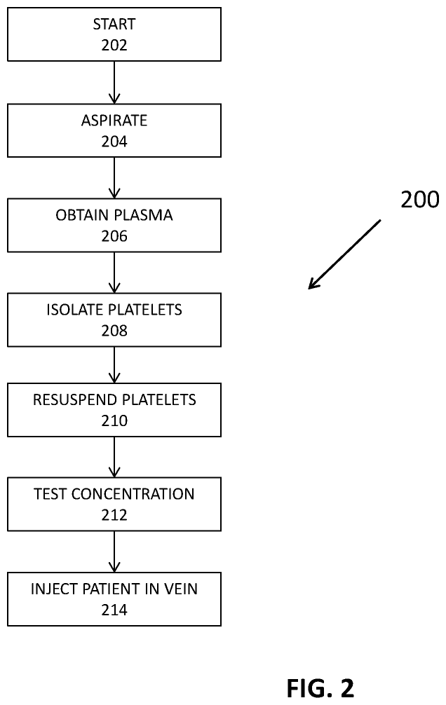

Referring now to FIG. 2, a PRP treatment method 200 is provided. At step 202 the method begins. In one embodiment of the treatment protocol, whole blood is aspirated from a patient at step 204. Next, at step 206, the whole blood is concentrated by obtaining a plasma fraction of the whole blood and isolating the platelets therefrom at step 208. Then the platelets are re-suspended at step 210 and the concentration of the final concentrate is tested at step 212. Finally, the PRP concentrate is administered via intravenous injection under time-controlled administration (approximately 1 mL per second) at step 214. Prior to IV injection, careful filtering of any impurities was undertaken to the PRP concentrate. Dose specific PRP-IV administration is indeed novel due to PRP being historically delivered only at the injury site. The novel aspect of the PRP-IV administration is the theoretical homing phenomenon whereby cells migrate to the organ of their origin or to repair. With neurodegenerative diseases, there are many underlying repair needs. The body releases platelets during times of repair. The current IV circulatory repair approach allowed high concentrations of platelets to home to various sites of repair before orchestrating a paracrine repair process and release of platelet growth factors. The current treatment protocol approach proved this as evident by patients reporting more than one improvement in neurological functions. In contrast to historic PRP site injections, growth factors are released immediately and focused to a specific injury. Again, this proved evident as the two patients who received site injections did not report any other benefits than the injury site pain relief. Therefore, PRP-IV administration promoting systemic circulatory repair proved beneficial.

In another aspect, the proposed treatment protocol adhered to a specific minimum dose concentration for each patient. Currently, there are no devices for PRP cell therapy that provide a method of quality control and standardization like the Harbour Cell Software™. In the absence of such device and standardization, physicians are either delivering suboptimal treatments or potentially inhibitory treatments. The lack of standardization and quality control inhibits the advancement of cell therapy knowledge. For example, a concentration of 3 billion platelets per mL may be inhibitory to tenocyte and neo-vascularization behavior. Knowing the optimal concentration range is not helpful unless the amount of injected PRP volume, the concentration machine used, the starting volumes used to concentrate, and/or the absolute value of platelets is also known. Aspirating the same amount of blood in each patient is not a scientifically sound way to reach the optimal concentration range. For example, if one patient has a baseline of 150,000 platelets/μL, and another has a baseline of 350,000 platelets/μL, then aspirating the same volume creates a significant variable. The current treatment protocol embodiment uses the Harbour Cell Software™ to eliminate the inherent variabilities, deliver consistent doses, and prohibit inhibitory concentrations.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications, and substitutions without departing from the spirit and scope of the invention.

What is claimed is:

1. A method of treating a neurological disorder comprising:
identifying a neurological disorder in a patient;
intravenously injecting about 7 mL of a platelet-rich plasma composition having a concentration of at least about 2×10⁶ platelets/μL into the patient to produce neurological improvements in the patient;
wherein the platelet-rich plasma composition is injected into a vein of the patient at a rate of about 1 mL/second; and
wherein the platelet-rich plasma composition does not include an activating agent.

2. The method of claim 1, wherein the concentration of the platelet-rich plasma composition is about 3×10⁶ platelets/μL.

3. The method of claim 1, wherein the platelet-rich plasma composition does not include a dilutant.

4. The method of claim 1, wherein the platelet-rich plasma composition does not include a saline dilutant.

5. The method of claim 1, wherein the platelet-rich plasma composition does not include an anticoagulant.

6. The method of claim 1, further comprising preparing the platelet-rich plasma composition from whole blood of the patient.

7. The method of claim 6, wherein preparing the platelet-rich plasma composition from the whole blood comprises the steps of:
obtaining a plasma fraction from the whole blood;
isolating platelets from the plasma fraction;
resuspending the platelets in a reduced amount of plasma; and
wherein an activator of the platelets is not added to the platelet-rich plasma composition.

8. The method of claim 1, further comprising testing the concentration of the platelet-rich plasma composition prior to injection.

9. A medical treatment protocol for treating symptoms of a neurological disorder in a human comprising:
determining a baseline platelet concentration of a blood sample of a patient;
determining a volume of blood to aspirate from the patient for a platelet-rich plasma treatment based at least in part on (a) a concentration target of 2×10⁶ platelets/μL, (b) the baseline platelet concentration, and (c) a treatment volume target of 7 mL of final concentrate;
aspirating the volume of blood from the patient;
concentrating the aspirated blood to obtain a treatment volume of at least about 7 mL of the final concentrate having a concentration of at least about 2×10⁶ platelets/μL; and
injecting the final concentrate into the patient intravenously at a rate of about 1 mL/second thereby alleviating at least one symptom of a neurological disorder.

10. The protocol of claim 9, wherein the concentration of the final concentrate is about 3×10⁶ platelets/μL.

11. The protocol of claim 9, wherein the final concentrate does not include a dilutant.

12. The protocol of claim 9, wherein the final concentrate does not include a saline dilutant.

13. The protocol of claim 9, wherein the final concentrate does not include an anticoagulant.

14. The protocol of claim 9, wherein concentrating the aspirated blood comprises the steps of:
obtaining a plasma fraction from the aspirated blood;
isolating platelets from the plasma fraction;
resuspending the platelets in a reduced amount of plasma; and
wherein an activator of the platelets is not added to the final concentrate.

15. The protocol of claim 9, further comprising testing the concentration of the final concentrate prior to injection.

16. A method for administering a medical treatment protocol comprising:
determining a baseline platelet concentration of a blood sample of a patient;
receiving an indication of a treatment volume of concentrate to be used in a cell-therapy treatment;
calculating an aspiration volume of blood to be aspirated for the cell-therapy treatment to achieve a platelet concentration target range of 2×10⁶ platelets/μL, based at least in part on the baseline platelet concentration for the patient and the indicated treatment volume of platelet-rich plasma concentrate;
aspirating the volume of blood from the patient;
concentrating the aspirated blood to obtain a treatment volume of at least about 7 mL of the platelet-rich plasma concentrate having a concentration of at least about 2×10⁶ platelets/μL; and
injecting the platelet-rich plasma concentrate into the patient intravenously at a rate of about 1 mL/second.

17. The method of claim 16, wherein the concentration of the platelet-rich plasma concentrate is about 3×10⁶ platelets/μL.

18. The method of claim 16, wherein concentrating the aspirated blood comprises the steps of:
obtaining a plasma fraction from the aspirated blood;
isolating platelets from the plasma fraction;
resuspending the platelets in a reduced amount of plasma; and
wherein an activator of the platelets is not added to the platelet-rich plasma concentrate.

19. The method of claim 16, wherein the platelet-rich plasma concentrate does not include a saline dilutant.

20. The method of claim 16, wherein the platelet-rich plasma concentrate does not include an anticoagulant.
